(19) United States
(12) Plant Patent Application Publication
(10) Pub. No.: US 2017/0079172 P1
(43) Pub. Date: Mar. 16, 2017

(54) Kiwifruit plant named 'Yang's Golden Red No. 1'

(50) ;

(71) Applicants: Shengmou Yang, Yangzhou (CN); Xiaoqin Jiang, Yangzhou (CN); Ying Lv, Yangzhou (CN); Jian Yang, Yangzhou (CN)

(72) Inventors: Shengmou Yang, Yangzhou (CN); Xiaoqin Jiang, Yangzhou (CN); Ying Lv, Yangzhou (CN); Jian Yang, Yangzhou (CN)

(21) Appl. No.: 14/756,546

(22) Filed: Sep. 16, 2015

(51) Int. Cl. A01H 5/08

(52) U.S. Cl. PLT/156

(57) ABSTRACT

A kiwifruit plant particularly distinguished by cylindrical fruit shape with a concave stylar end, pale yellow fruit flesh with red color radiating from the axial ovary, early fruit maturity and an average fruit weight of 90 to 94 grams is described.

Genus and species: Actinidia chinensis.

Variety denomination: ‘Yang's Golden Red No. 1’.

### BACKGROUND OF THE INVENTION

The present invention relates to a new and distinct variety of Actinidia chinensis (kiwifruit) hereinafter referred to by the cultivar name ‘Yang's Golden Red No. 1’ or the ‘new plant’. ‘Yang's Golden Red No. 1’ was selected from a population of seedlings derived from crossing two kiwifruit selections ‘Hongyang’ (U.S. Plant patent application Ser. No. 11/645,130), the female; and ‘Male No. 13’ (unpatented), the non-fruiting male; using controlled pollination in the course of a planned kiwifruit variety breeding program. The new plant was selected on 9 Sep., 2004 at Huaisi Town, Yangzhou, China.

The new plant has been asexually propagated by grafting in Huaisi Town, Yangzhou, China and the unique combination of characteristics of the new plant has been found to be reproducible and stable in successive generations of propagation. In order to obtain true-to-type clones of the initial plant, asexually propagated plants were obtained by grafting dormant buds from the original seedling onto Actinidia deliciosa rootstock.

Plant Variety Rights for ‘Yang's Golden Red No. 1’ were granted in China on 1 Nov., 2014 and assigned the grant number CNA20110642.7.

### SUMMARY OF THE INVENTION

The following are the most outstanding and distinguishing characteristics of the new plant when grown under normal horticultural practices in Huaisi Town, Yangzhou, China:
1. Cylindrical fruit shape with concave stylar end
2. Pale yellow fruit flesh with red color radiating from the axial ovary
3. Early maturing fruit
4. Average fruit weight of 90 to 94 grams

### DESCRIPTION OF THE PHOTOGRAPHS

The new plant is illustrated by the accompanying photographs which show in full color typical specimens of the plant habit, foliage, fruit and flowers of the new variety ‘Yang's Golden Red No. 1’. The colors shown are as true as can be reasonably obtained by conventional photographic procedures. The photographs include plants approximately five years after grafting onto Actinidia deliciosa ‘Hayward’ (unpatented) rootstock in optimum conditions in Huaisi Town, Yangzhou, China.

FIG. 1 shows typical fruit on the vine

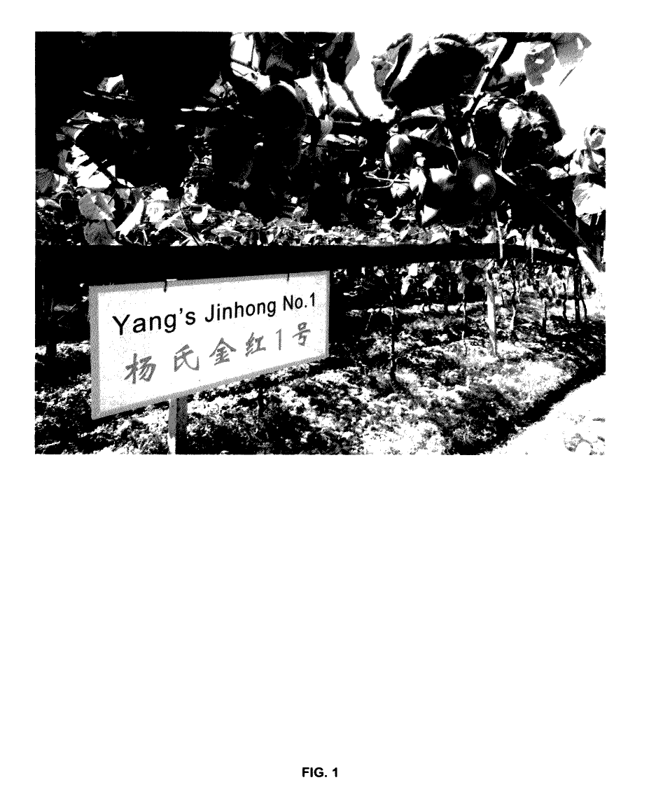

FIG. 2 shows a close up of typical fruit on the vine

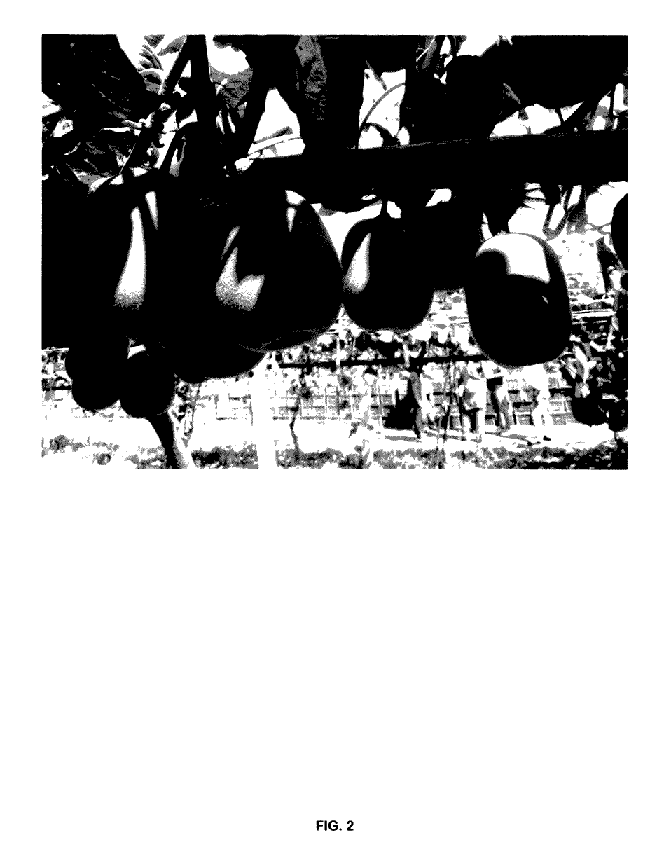

FIG. 3 shows fruit in longitudinal section and cross section in the studio

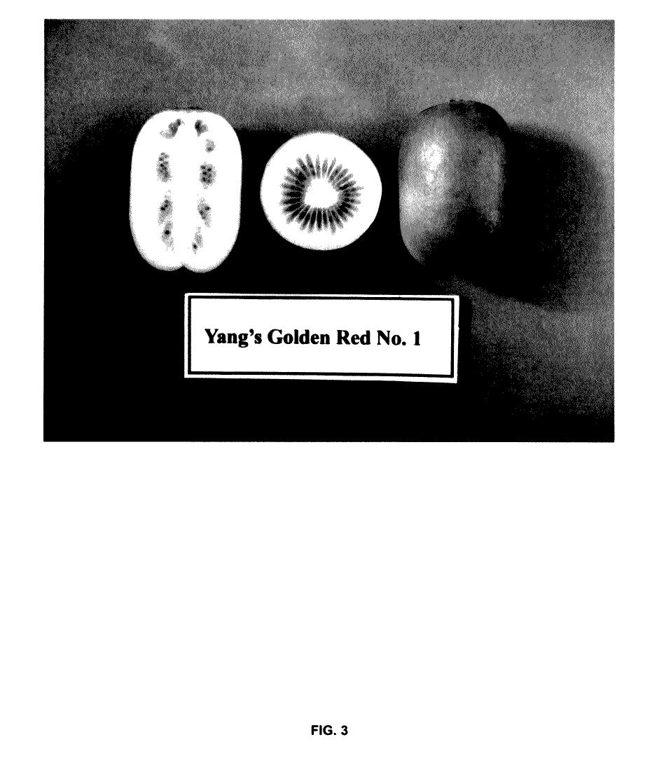

FIG. 4 shows upper surface of mature leaves in the studio

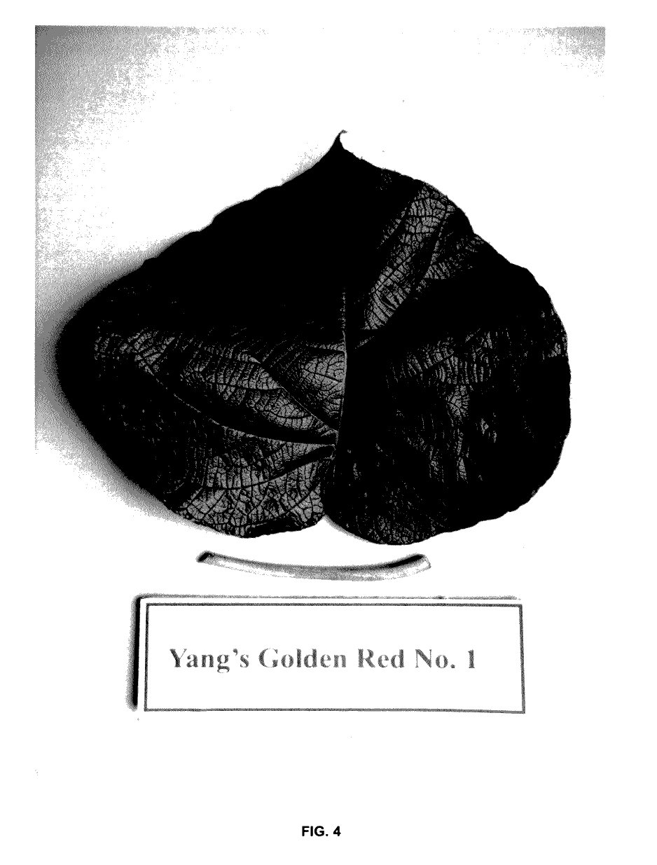

FIG. 5 shows lower surface of mature leaves in the studio

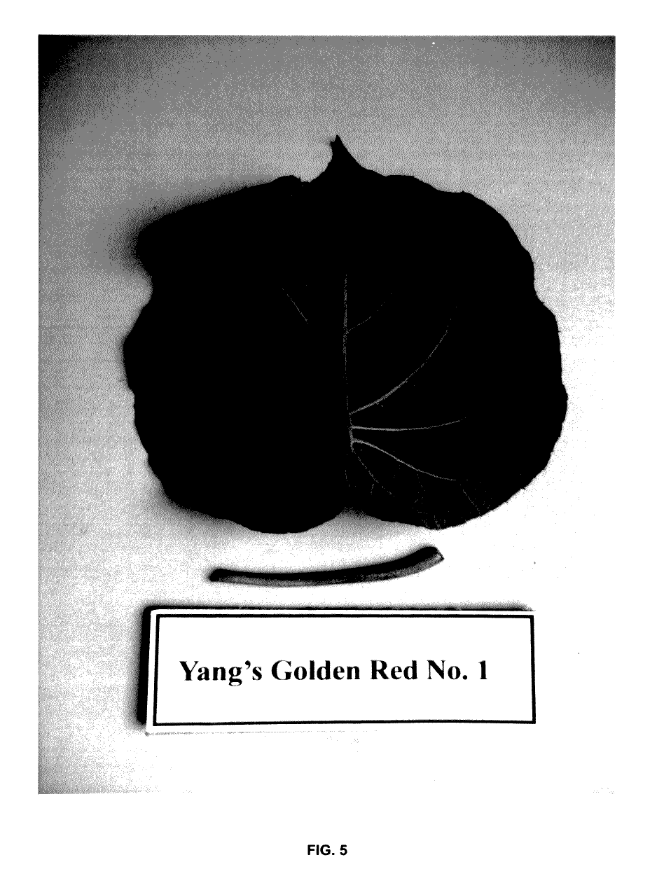

FIG. 6 shows young leaves and one year old shoots on the vine

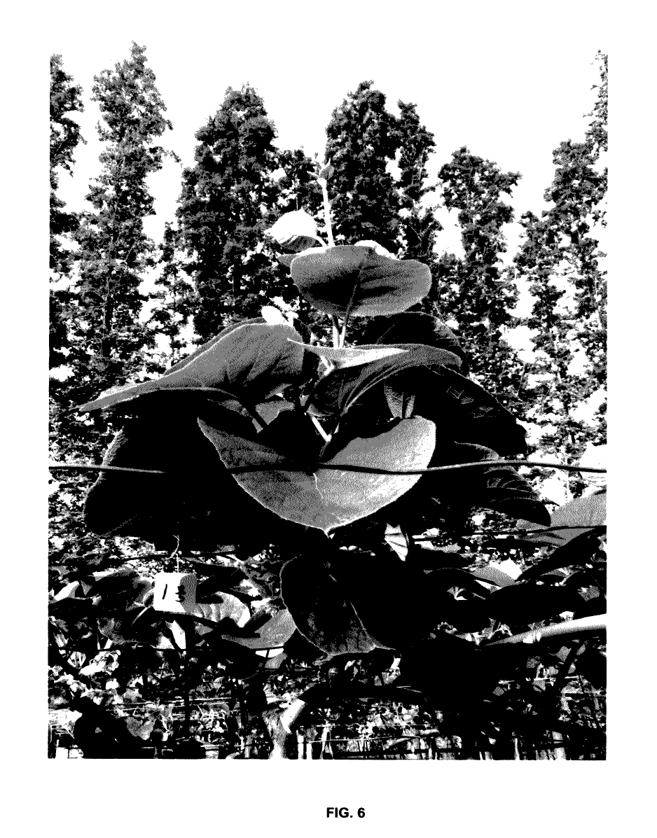

FIG. 7 shows flowers on the vine

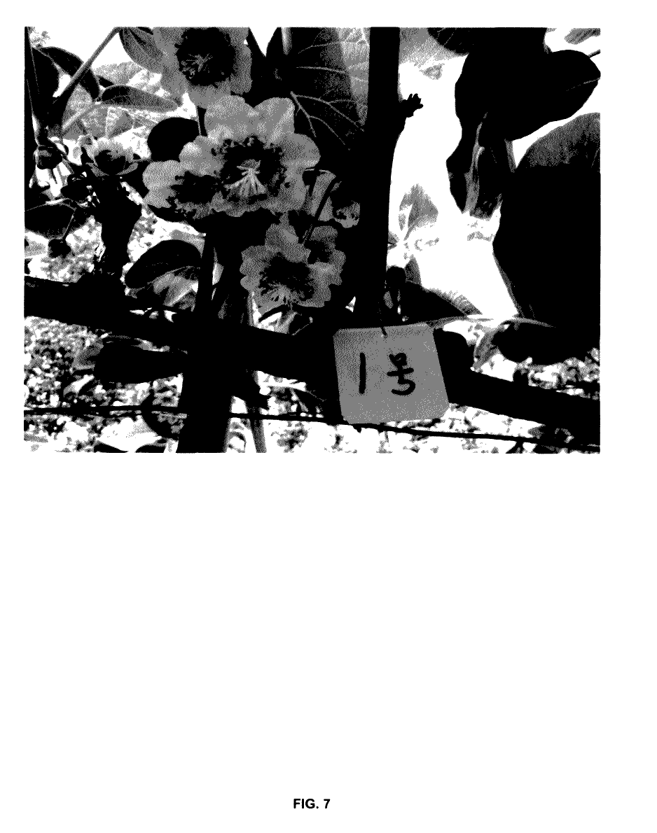

FIG. 8 shows typical flower, flower bud, petal and pedicel in the studio

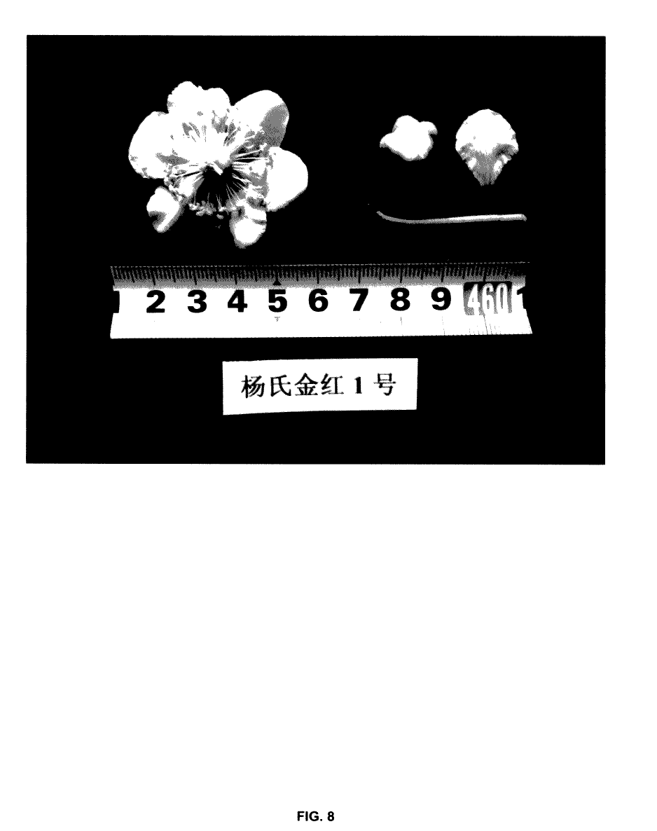

FIG. 9 shows typical flower, flower bud, petal and pedicel in the studio

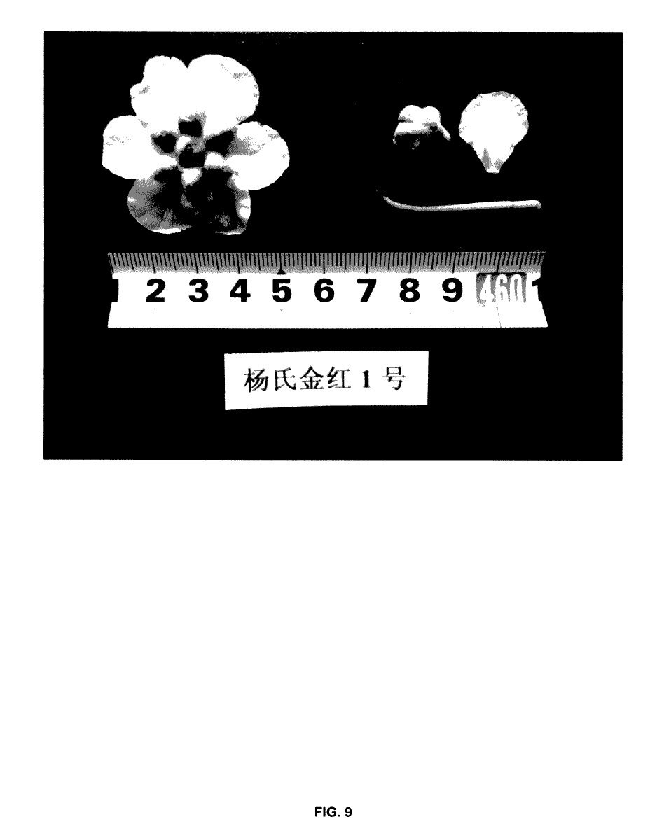

### DESCRIPTION OF THE NEW CULTIVAR

The following detailed description sets forth the distinctive characteristics of ‘Yang's Golden Red No. 1’. The following observations, measurements and values were made on five year old vines grafted onto Actinidia deliciosa ‘Hayward’ (unpatented) rootstock, and managed under standard orchard practice in Huaisi Town, Yangzhou, China. Random measurements of each characteristic were obtained from a sample of 50 plants unless otherwise stated.

Horticultural terminology is used in accordance with UPOV guidelines for kiwi. All dimensions are in millimetres, and all weights are in grams (unless otherwise stated). Certain characteristics of this variety, such as growth and color, may change with changing environmental conditions (e.g., light, temperature and moisture), nutrient availability, rootstocks, or other factors. Color readings were taken in natural light. Color references are primarily to the Fourth edition, 2001 of the Colour Chart published by The Royal Horticultural Society, London (R.H.S.), except where general colours of ordinary significance are used.

### DETAILED BOTANICAL DESCRIPTION

Classification:
   Family.—Actindiaceae.
   Botanical name.—Actinidia chinensis.
   Common name.—Kiwifruit.
Parentage:
   Female parent.—A kiwifruit selection ‘Hongyang’ (U.S. Plant patent application Ser. No. 11/645,130).
   Male parent.—A kiwifruit selection ‘Male No. 13’ (unpatented).
Plant:
   Plant habit.—female plant of twining habit and medium vigour.
   Average plant size.—plants are grown on a standard pergola structure; each plant was pruned to occupy a canopy area of approximately 5 m².
   Ploidy.—tetraploid.
   Propagation.—grafting of dormant buds onto rootstock.
Shoots:
   Texture of young shoots.—smooth.
   Colour of young shoots.—green, RHS 141C.
   Texture of mature shoots.—smooth with slightly raised lenticels.
   Colour of mature shoots.—brown, RHS 200D.
   Diameter of mature shoots.—10 mm to 18 mm.
   Lenticels on shoots.—Quantity/density: many Length: 1 mm to 13 mm Shape: narrowly elliptical Colour: greyed-white, RHS 156C.
Leaves:
   Quantity per one metre of mature shoot.—10 to 15.
   Arrangement.—alternate.
   Length (mature leaf).—133 mm to 152 mm.
   Width (mature leaf).—118 mm to 136 mm.
   Shape.—cordate.
   Apex.—obtuse.
   Base.—cordate, overlapping.
   Margin.—finely serrate.
   Leaf spines.—approximately 1 mm in length.
   Texture.—Upper surface: slightly puckered, non-pubescent Lower surface: no pubescence.
   Colour.—Young leaves, upper surface: green, RHS 138A Young leaves, lower surface: near green, RHS 138B Mature leaves, upper surface: green, RHS 137B Mature leaves, lower surface: near green, RHS 138C.
   Venation.—Pattern: reticulate Colour, upper surface: near green, RHS 138A Colour, lower surface: near green, RHS 138C.
   Petiole (leaf stem).—Length: 120 mm to 140 mm Diameter (cross section): 3.2 mm to 3.9 mm Colour: near green, RHS 138A.
   Texture.—smooth, no pubescence.
Inflorescence:
   Inflorescence type.—one king flower with occasional 1-2 smaller side flowers.
   Natural flowering season.—Full bloom during late April.
   Flower number per square metre.—70 to 100.
   Fragrance.—light.
   Longevity of viable flowers on the plant.—5 to 6 days.
   Pedicel (stalk holding each flower).—Strength: moderate Length: 40 mm to 60 mm. Colour: green, RHS 138C Texture: sparsely pubescent.
   Flowers.—Flower bud (diameter): 12 mm to 16 mm Flower bud (length): 6 mm to 7 mm Colour of bud: green, RHS 139C Shape of bud: round to slightly elliptical Diameter of individual flower: 30 mm to 45 mm Arrangement of petals: overlapping.
   Petals.—Quantity: 6 to 7 Length: 20 mm to 25 mm Width: 15 mm to 17 mm Shape: oblanceolate Margin: undulate Type of colouration: Colour, primary when fully opened: white, 155C Colour, base of petals and centre when fully opened: green white, 157C.
   Sepals.—Quantity: 6 to 7 Length: 5 mm to 8 mm Width: 4 mm to 7 mm Shape: oblanceolate Colour: green, RHS 13813 Texture: tomentose.
   Peduncles.—Length: 38 mm to 45 mm Colour: green, RHS 138C Texture: sparsely pubescent.
Reproductive organs:
   Styles.—Quantity: 30 to 40 Attitude: semi erect Colour: white, RHS 155C.
   Stamens.—Quantity: 60 to 80.
   Anthers.—Colour: orange, RHS 24A.
   Ovary.—(king flower). Length: 8-9 mm. Shape: ellipsoid Colour: white, RHS 155C Texture: densely pubescent.
   Fruit.—Length: 48 mm to 55 mm Diameter: 39 mm to 45 mm Weight range: 85 g to 100 g Average weight: 90 to 94 g.
   Skin.—Texture: sparsely pubescent Colour at harvest: yellow green, RHS 152C Adherence to flesh: strong.
   Shape.—Longitudinal: cylindrical. Cross section: circular Stylar end: concave Shoulder: squared.
   Calyx ring.—present Expression: strong.
   Stalks.—Length: 38 mm to 45 mm Diameter: 2 mm to 3.1 mm Texture on mature fruit: smooth Colour on mature fruit: green, RHS 139C.
   Lenticels.—Quantity: sparse Colour: yellow green, RHS 152C Size: 1-2 mm (diameter).
   Pericarp.—Outer colour, early: green, RHS 141C Outer colour, mature: yellow green, RHS 152C Inner colour, mature: green yellow, RHS 1D; colour radiating around seeds red, RHS 43C.
   Core of fruit.—Colour, mature: white, RHS 155C Shape: transversely elliptical Quantity of locules: 30 to 42 Quantity of Seeds: 40 to 50 per transverse slice through middle of fruit Colour of seeds: greyed orange, RHS 165B.
   Flavour.—Taste: sweet Dry matter at harvest: 17% to 19% Brix level at harvest: 7% Brix level at consumption: 17% to 20%.
   Storage.—20 weeks at 1 degree Celsius.
   Overall fruit cropping per hectare.—25 to 40 tons.
Rootstock: ‘Yang's Golden Red No. 1’ is commonly grown on Actinidia deliciosa seedling rootstock
Vegetative bud break:
   Timing.—mid-March.
   Quantity of buds breaking.—75% to 85%.
   Crop load.—2.5 kg to 3 kg per square meter average.
   Leaf senescence.—late November.
Disease resistance: No observations made.
Insect resistance: No observations made.
Temperature tolerance: Tolerates low temperatures to approximately −5° C. without negative effects, tolerates high temperatures to approximately 35° C. without negative effects.

### COMPARISON WITH PARENTAL AND KNOWN CULTIVARS

When compared to the female parent ‘Hongyang’ (U.S. Plant patent application Ser. No. 11/645,130); ‘Yang's Golden Red No. 1’ has a greater number of lenticels on the shoots, longer petioles, longer fruit stalks and a heavier average fruit weight.

When compared to the male parent ‘Male No. 13’ (unpatented) which is a non-fruiting male pollinator; ‘Yang's Golden Red No. 1’ has brown mature stems with elliptical lenticels, cordate leaves and bears fruit; whereas ‘Chinensis Male No. 13’ (unpatented) has dark grey mature stems with circular lenticels, widely ovate leaves and does not bear fruit.

When compared to the commercial variety ‘Hort16A’ (U.S. Plant Pat. No. 11,066); fruit of ‘Yang's Golden Red No. 1’ is cylindrical in shape with a concave stylar end, squared shoulder and has pale yellow flesh with red color radiating from the axial ovary; whereas fruit of ‘Hort16A’ (U.S. Pat. No. 11,066) is ovoid in shape with a strong pointed stylar end, rounded shoulder and has golden yellow flesh.

When compared to the commercial variety ‘Yang's Golden Red No. 50’ (U.S. Plant patent application Ser. No. 14/120,479); ‘Yang's Golden Red No. 1’ has smaller leaves, fruit has a concave stylar end as opposed to a slightly blunt protruding stylar end, fruit matures 3-4 weeks earlier and has fewer seeds and a lower average weight.

I claim:

1. A new and distinct cultivar of kiwifruit plant as shown and described herein.
